(19) United States
(12) Patent Application Publication
(10) Pub. No.: US 2008/0119688 A1
(43) Pub. Date: May 22, 2008

(54) Adjustably compressive female incontinence device

(76) Inventors: Antoine Jean Henri Robert, Rio de Janeiro (BR); Joao Luiz Schiavini, Rio de Janeiro (BR)

Correspondence Address: LAURA N. TUNNELL, P.O. BOX 6003, SANTA BARBARA, CA, 93160, US

(21) Appl. No.: 11/582,635

(22) Filed: Oct. 17, 2006

(51) Int. Cl. A61F 2/02

(52) U.S. Cl. 600/31; 600/29

(57) ABSTRACT

An adjustable incontinence device for implantation within the body of a female and a method for implanting and using the device for controlling urinary incontinence. The device includes an elastomeric tube having an elongate inflatable balloon affixed to one end thereof and an implantable inflation port affixed to the opposing end of the tube. At least a portion of the balloon in contact with the periurethral tissue is covered with a polyurethane foam. The device is implanted beneath the skin with the balloon disposed anterior and adjacent to the urethra and the tube threaded beneath the skin and attached to the inflation port which is implanted at a transdermally accessible point remote from the balloon. Fluid is introduced into the injection port by transdermal injection and conducted to the interior chamber of the balloon to inflate the balloon and compress the underlying urethra.

### BACKGROUND OF THE INVENTION

1. Field of the Invention

The present invention relates to a device for the treatment of stress incontinence and, more particularly, to an implantable, adjustable device for compressing the urethra of a woman to treat urinary incontinence.

2. Prior Art

The problem of urinary incontinence is common and growing. For example, in North America, incontinence affects more than twenty million people and more than half the admissions to chronic centers and an annual expense of a billion dollars for absorbent devices are attributed to it. Moreover, the problem is increasing with the expectation of greater longevity of the population.

A distinction must be made as to whether the problem occurs in an active healthy person or in someone who is institutionalized in either a chronic care facility or in a nursing home. The treatment approaches will necessarily be different. In the case of the institutionalized person suffering from urinary incontinence, they are often unable to have any surgical procedure to correct their condition so that non-invasive (non-surgical) approaches are required. The common practice in most nursing homes at present is to have the patients fitted with an absorbent diaper-like material. For various reasons, most nursing homes will not accept patients with indwelling catheters.

The involuntary loss of urine may occur by means of two mechanisms which presently require different surgical techniques for their correction. The most common cause is the descent or dropping of the neck of the urinary bladder and of the urethra (hypermobile urethra). The other is due to the fact that the urethra has lost its closure capacity (deficiency of the internal sphincter). In men, incontinence is produced by lesion of the external sphincter in the course of operations on the prostate.

Many devices have been designed to deal with the problem of urinary incontinence and the difficulties associated with the use of these devices are well known. The basic problem found in many females with urinary incontinence is that there is a descent of the bladder neck and an associated wide open bladder neck and upper third of urethra, the so-called funnel-shaped urethra. In accordance with the current state of the art, to correct the incontinence without surgery, a device which either occludes the urethra or elevates the bladder neck must be employed. Many of the prior art devices are designed to be placed in the vagina but retaining the device has been one of the main problems associated with their use. In an effort to increase the obstruction to the flow of urine, various other techniques have been used. More recently periurethral injections with various compounds have been used. The purpose of these injections is to obliterate the lumen of the urethra and thus reduce the urinary incontinence. Some of the substances injected include periurethral Teflon, injections of collagen and more recently periurethral injections of autogous fat. In some instances, urethral catheterization has been used to control incontinence.

More recently, there have been various urethral plugs designed for inserting in the urethra to occlude the lumen. These plugs are disposable and have to be re-inserted after each voiding. Some of the plugs are retained by means of a balloon arrangement and these all carry the risk of urethral irritation and infection.

Previous prosthetic devices have attempted to achieve normal bladder support by applying pressure to the anterior wall of the vagina and tissue disposed posterior to the urethra. Such applied pressure does not provide lateral alignment of the urethra and may serve to exacerbate any lateral urethral deviation. Moreover, when such a device exerts pressure on the tissue in excess of 32 mm Hg, the capillaries close, cutting off the cellular blood supply and eventually resulting in necrosis and erosion of the adjacent tissue, manifested as vaginal pressure ulcers. Even where such devices do not damage the tissue by exerting excessive pressure, the pressure they do cause results in occlusion of the urethra, setting up a local irritation and attendant patient discomfort.

Scetbon, in U.S. Pat. No. 6,478,727, discloses a device for treating urinary stress incontinence in women that includes a flexible elongate tape for supporting a urethra and a flat protective sheath enveloping the tape; and a puncturing needle with an active distal end and a proximal end connected to a first end of the flexible and elongate mechanism, wherein the proximal end of the puncturing needle is connected to a first end of the flexible and elongate mechanism by an intermediate traction element, a second end of the flexible and elongate mechanism being free. A method for treating urinary stress incontinence in a woman suffering from urinary stress incontinence is also provided including (a) forming an opening in an anterior vaginal wall; (b) creating, from two small suprapubic incisions formed in the abdominal wall, a right track and a left track from the abdominal skin to the opening formed in the anterior vaginal wall; (c) using a needle and an intermediate traction element to follow one of the tracks and following the other track with at least a needle; (d) verifying by cystoscopy that the paths of the tracks are outside the bladder and the urethra; (e) using a support tape surrounded by a plastic sheath to follow the tracks by passage under an inferior surface of the urethra; (f) adjusting a loop formed by the sheathed tape under the inferior surface of the urethra; (g) removing the sheath by pulling the sheath toward the outside of the woman's body through the small suprapubic incisions; and (h) leaving the tape implanted from the first to the second incision and around the urethra to support the urethra.

Polyak, in U.S. Pat. No. 5,518,504, discloses an implantable artificial sphincter system for reversibly occluding a body passageway, preferably the urethra, enables the body passageway to be opened and closed in sequence due to the action of a lifting and lowering device which is adapted to be in integral contact with or disposed about the body passageway. An actuating device is operably connected to the lifting and lowering device, and a pump capable of being volitionally operated is in fluid communication with the actuating device to provide a pressurized flow of fluid into the actuating device.

Gil-Varnet, in U.S. Pat. No. 6,117,067, discloses a device for adjusting the height and supporting internal anatomical organs. The device comprises a chamber having elastic properties such as a balloon, the volume of which varies in accordance with the amount of fluid in the balloon. To increase or decrease the volume, a inflation/deflation port connected by a tube to the balloon is provided which is adapted to be located near the skin of the patient. A hypodermic needle can extract or introduce fluid into the inflation port to change the volume of the interior chamber of the balloon. A thread is connected to one base of the chamber and the other base is adapted to sit on an anatomical region in the body. The thread is adapted to surround the selected organ whereby changes in the volume of the chamber changes the distance between the bases thereby to move the organ.

Most implantable female incontinence devices operate by lifting either the neck of the bladder or the urethra. There is relatively little tissue between the urethra and the vagina into which the lifting member must be disposed. Accordingly, as mentioned above, there is a high incidence of erosion of either the urethra or the vagina following implantation of a device posterior to the urethra. There is a need for an adjustable incontinence device and a method for using the device that reduces the incidence of erosion of the vaginal wall and/or the urethra following implantation thereof. In addition, it is desirable that such an implantable incontinence device has relatively few parts, is adjustable following implantation and can be implanted endoscopically.

### SUMMARY

It is an object of the present invention to provide an adjustable implantable incontinence device and a method for implanting an inflatable portion of the device anterior to the urethra of a woman to compress the urethra downwardly for the treatment of urinary incontinence.

The features of the invention believed to be novel are set forth with particularity in the appended claims. However the invention itself, both as to organization and method of operation, together with further objects and advantages thereof may be best understood by reference to the following description taken in conjunction with the accompanying drawings.

### BRIEF DESCRIPTION OF THE DRAWINGS

FIG. 1 is an anatomical longitudinal section of a human female body illustrating the subcutaneous placement of an incontinence device anterior to the urethra in accordance with the present invention.

FIG. 2 is a perspective view of an implantable adjustable female incontinence device of the present invention.

FIG. 3 is a cutaway anterior anatomical view of the genitourinary tract of a woman illustrating the implantation of the inflatable portion of an adjustable incontinence device anterior to the urethra in accordance with the method of the present invention.

### DESCRIPTION OF THE PREFERRED EMBODIMENTS

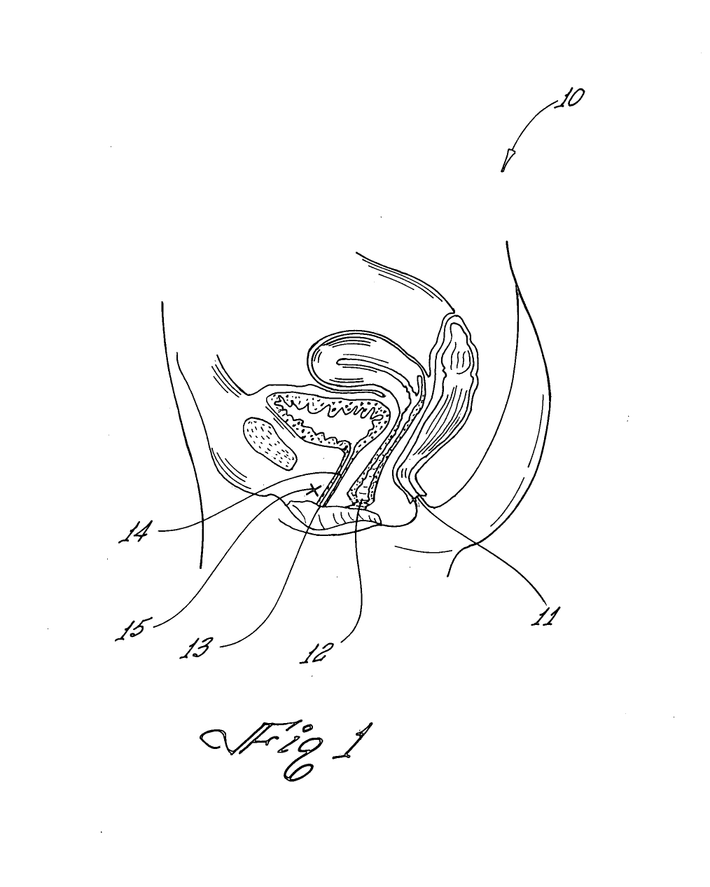

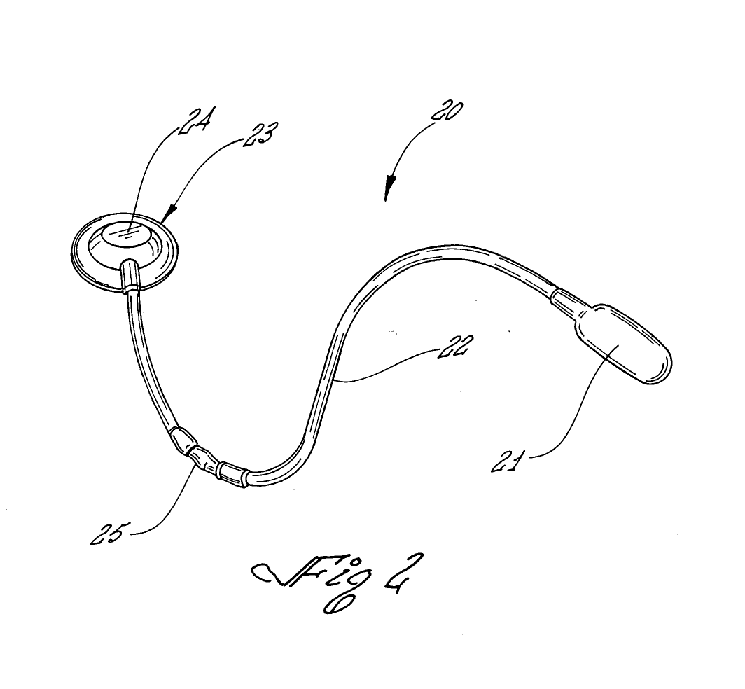

FIG. 1 is an anatomical longitudinal section of the abdominal portion of a human female body 10 illustrating the position X of an inflatable balloon portion 21 of an incontinence device 20 (FIG. 2) anterior to the urethra 14. FIG. 1 illustrates the relative position of the rectum 11, the vagina 12 and the external meatus 13 of the urethra 14. The tissue 15 between the vagina 12 and the urethra 14 is relatively thin and the implantation of an inflatable device in tissue 15 can cause erosion of the vaginal wall and/or urethra 14 adjacent thereto. In accordance with the present invention, the inflatable balloon portion 21 (FIG. 2) of the device 20 is implanted in tissue X anterior to the urethra 14. The implantation of the balloon 21 in the tissue indicated at X enables the inflatable portion 21 to be spaced from the urethra to avoid erosion thereof while enabling constriction of the urethra by compressing tissue anterior thereto when the balloon 21 is inflated.

Turning now to FIG. 2, the implantable adjustable female incontinence device 20 comprises an elongate inflatable balloon 21 having one end of a length of elastomeric tubing 22 attached thereto in leak-proof engagement therewith and an inflation port 23 attached to the opposing end of the tubing 22. The tubing connector 25 forming attachment between the tubing 22 and the inflation port 23 is similarly leak-proof. The inflation port 23 has an elastomeric septum 24 affixed thereto that is self-sealing to the track of a hollow bore needle (not shown) when the septum 24 is penetrated thereby. The inflation port 23 has an interior chamber underlying the septum 24 that is in fluid communication with the interior chamber of the balloon 21 by means of an axial lumen in the tubing 22 and tubing connector 25.

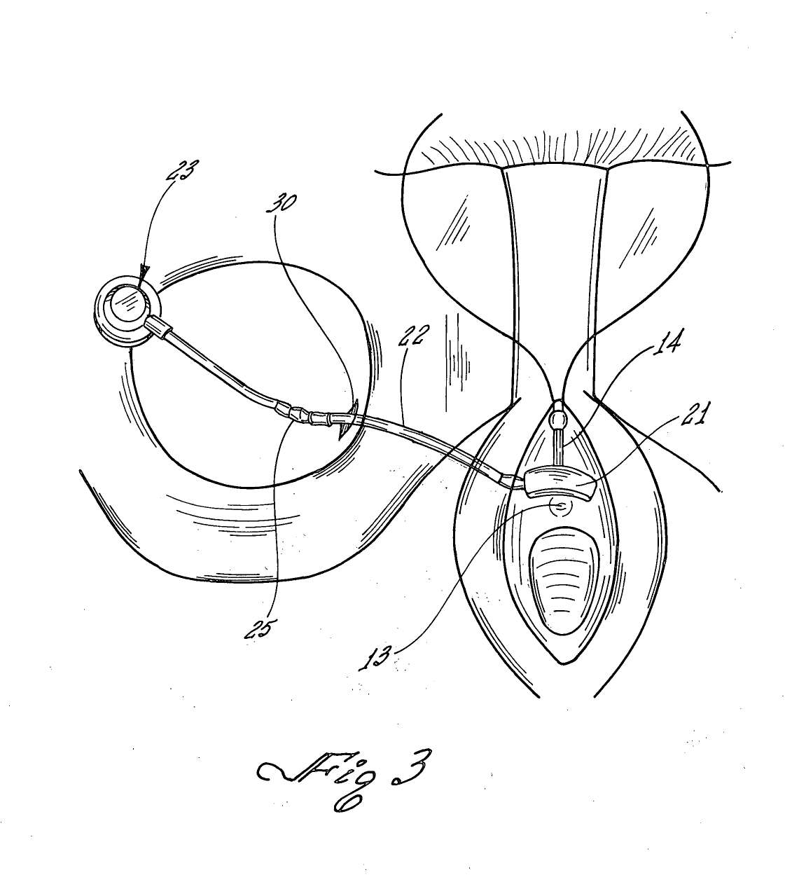

The method for surgically implanting the device 20 beneath the skin of an incontinent female patient is an important feature of the invention. FIG. 3 is a cutaway anterior anatomical view of the genitourinary tract of a woman illustrating the implantation of the inflatable portion of an adjustable incontinence device anterior to the urethra in accordance with the method of the present invention. As with similarly shaped devices, the device 20 (exclusive of the inflation port 23) can be implanted endoscopically. Alternatively, the balloon 21 and tubing 22 can be implanted by: (a) placing the balloon 21 and tube 22 in the bore of a needle having a hole at or near the distal (sharp) tip thereof; then (b) making a small incision 30 through which the needle bearing the balloon can be introduced beneath the skin and positioning the hole in the distal end of the needle anterior to the urethra 14; (c) extruding the balloon 21 and tubing 22 through a hole at or near the tip of the hollow bore needle; (d) removing the needle: (e) surgically implanting the inflation port 23 beneath the skin at a convenient and accessible site such as over the abdominal muscles or the iliac crest as shown in FIG. 3; then connecting the tubing 22 to the inflation port 23 by tubing connector 25; then (f) inflating the balloon 21 by injecting a fluid into the inflation port 23. The positioning of the balloon anterior to the urethra permits more tissue to be interposed between the balloon and the urethra than is possible by implanting the inflatable compressive (or lifting) device between the urethra and the vagina in accordance with the method of the prior art. The present method of implantation of the balloon 21 allows additional periurethral tissue to be interposed between the balloon and the urethra which reduces the chance of erosion of the urethra and obviates erosion of the vagina. In a preferred embodiment of the incontinence device, the surface of the balloon 21 that contacts periurethral tissue is advantageously covered with a layer of polyurethane foam to promote tissue ingrowth into the foam layer thereby anchoring the balloon in the desired position.

While particular embodiments of the present invention have been illustrated and described, it would be obvious to those skilled in the art that various other changes and modifications can be made without departing from the spirit and scope of the invention. For example, as stated above, it may be advantageous to texture at least a portion of the outer surface of the balloon, for example, by adhering a layer of open-celled polyurethane foam thereto, to enable tissue ingrowth and facilitate anchoring of the balloon in a position adjacent the urethra. It is therefore intended to cover in the appended claims all such changes and modifications that are within the scope of this invention.

What we claim is:

1. A device for treating urinary incontinence in a female comprising: (a) an inflatable balloon having an interior chamber and an outer tissue-contacting surface wherein said outer tissue-contacting surface has a layer of open-celled polyurethane foam adhered thereto; (b) an implantable inflation port having a septum on an outer surface thereof and integral therewith wherein said septum is self-sealing following penetration thereof by a hollow bore needle, and an interior chamber operable for receiving a fluid injected through said septum into said interior chamber of said inflation port; and (c) a single-lumen inflation tube connecting said inflatable balloon to said inflation port, said single-lumen inflation tube providing fluid communication between said interior chamber of said inflation port and said interior chamber of said balloon.

2. (canceled)

3. A method for treating urinary incontinence in a female comprising the steps of: (a) presenting a device comprising: (i) an inflatable balloon having an interior chamber and an outer tissue-contacting surface wherein said outer tissue-contacting surface has a layer of open-celled polyurethane foam adhered thereto; (ii) an implantable inflation port having a septum on an outer surface thereof and integral therewith wherein said septum is self sealing following penetration by a hollow-bore needle, and an interior chamber operable for receiving a fluid injected through said septum into said interior chamber of said inflation port; and (iii) a single lumen inflation tube providing fluid communication between said interior chamber of said inflation port and said interior chamber of said balloon; then (b) implanting said inflatable balloon anterior and adjacent to a urethra within said female and affixing said inflation port to tissue such that the inflation port overlies an anterior surface of a pelvic bone; then (c) implanting said inflation tube such that it provides fluid communication between said interior chamber of said inflation port and said interior chamber of said inflatable balloon; then (d) injecting a fluid into said interior chamber of said injection port thereby inflating said balloon to force tissue disposed between said balloon and the urethra downwardly against the urethra until the urethra is at least partially constricted.
